(19) United States
(12) Patent Application Publication
(10) Pub. No.: US 2015/0005552 A1
(43) Pub. Date: Jan. 1, 2015

(54) Crude Butadiene Pre-Treatment for Removal of High Furan Content

(71) Applicant: UOP LLC, Des Plaines, IL (US)

(72) Inventors: Steven L. Krupa, Fox River Grove, IL (US); Timothy D. Foley, Forest Park, IL (US)

(21) Appl. No.: 13/927,748

(22) Filed: Jun. 26, 2013

(51) Int. Cl. C07C 7/10; C07C 7/167

(52) U.S. Cl. CPC C07C 7/10; C07C 7/167
USPC 585/259; 585/839

(57) ABSTRACT

A process is presented for the purification of 1,3 butadiene. The process is for treating a butadiene stream from an oxidative dehydrogenation unit, where a butane stream is dehydrogenated, generating a butadiene rich stream. The butadiene rich stream is fractionated and passed through a butadiene recovery unit. Additional C4 compounds recovered from the fractionation bottoms stream are further processed for increasing yields of butadiene.

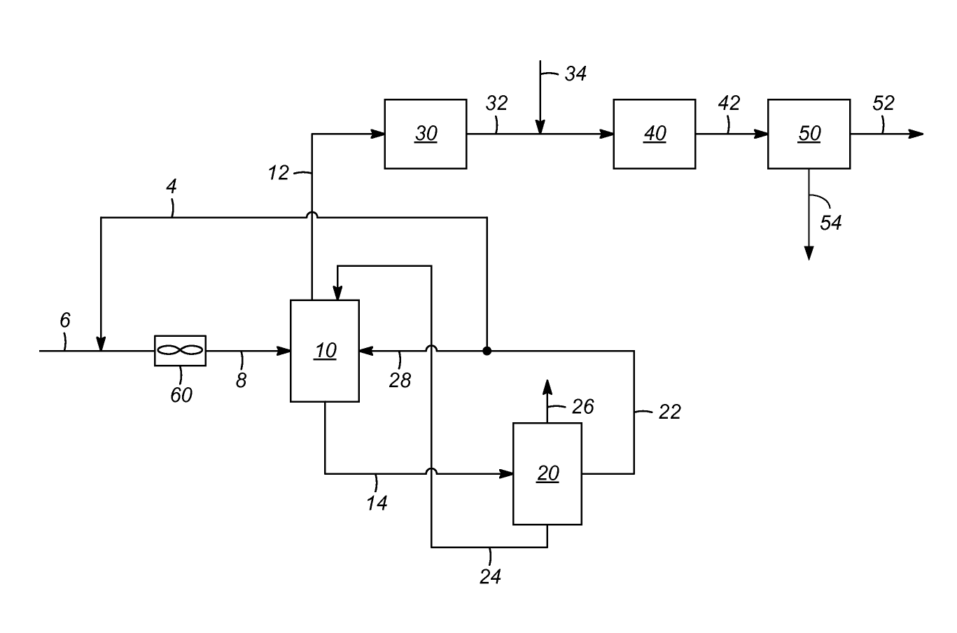

### FIELD OF THE INVENTION

The field of the invention relates to the dehydrogenation of hydrocarbons. Specifically, the invention relates to the purification process of a crude butadiene stream in the recovery of products from the dehydrogenation of light hydrocarbons using an oxidative dehydrogenation process.

### BACKGROUND OF THE INVENTION

1,3-Butadiene is an important monomer for the production of high molecular weight polymers. It is used extensively in the production of tires and other products in the automotive industry. It is also used extensively in the production of synthetic rubbers, such as styrene-butadiene rubber, nitrile-butadiene rubber, and styrene-butadiene latex. Polymer grade butadiene requires a high purity, and needs to be essentially free of active chemicals such as acetylenes, carbonyls, and contaminants such as sulfur and other heavy components. As used, hereinafter, the term butadiene will be used to refer to 1,3-butadiene.

Butadiene is usually a by-product recovered from the stream cracking of naphtha during the production of ethylene and propylene. Naphtha cracking, either through stream or catalytic cracking, produces a range of products, and is not optimized for the production of chemicals such as butadiene. Another route for the production of butadiene is through catalytic oxidative dehydrogenation of n-butane, which yields a higher concentration of butadiene, but also yields some undesirable side products that must be removed.

### SUMMARY OF THE INVENTION

The present invention provides a process for the removal of heterocyclic oxygenates from a hydrocarbon stream, where the heterocyclic oxygenates, and other oxygenates that are not sufficiently removable with a wash system that removes oxygenates such as aldehydes and ketones. The wash system is a bisulfite wash that will remove many of the oxygenates generated that are soluble in the bisulfite solution. The process is useful for the production of butadienes. Butadienes can be generated by oxidative dehydrogenation with a relatively high concentration, but also generates furan.

The process includes mixing the butadiene stream from the oxidative dehydrogenation process with a solvent to generate a mixed process stream. The mixed process stream forms a first process stream and is passed to an extraction unit with additional solvent for the removal of oxygenates, and in particular furan into the solvent. Water is passed to the extraction unit to facilitate the separation of the butadiene and solvent mixture into an aqueous phase and a non-aqueous phase. The non-aqueous phase forms a second process stream having a reduced oxygenate content, and the aqueous phase forms a third process stream with a high oxygenate content. The second process stream is passed for further processing to generate a purified 1,3-butadiene stream. The further processing includes a wash to remove residual aldehydes and ketones, and passing the washed second process stream to a selective hydrogenation unit for the selective hydrogenation of acetylenes to generate a fourth stream having reduced acetylenes content. The fourth process stream is passed to a 1,3-butadiene separation unit.

The third process stream is passed to a solvent recovery unit, where the oxygenates are separated from the third process stream and removed from the system. The solvent and water are recovered in the solvent recovery unit and recycled to the extraction unit. A preferred solvent is methanol.

Other objects, advantages and applications of the present invention will become apparent to those skilled in the art from the following detailed description and drawings.

### BRIEF DESCRIPTION OF THE DRAWING

The FIGURE is a process for the production of polymer grade 1,3-butadiene.

### DETAILED DESCRIPTION OF THE INVENTION

The production of butadiene can be performed with oxidative dehydrogenation of n-butane. The oxidative dehydrogenation process provides an attractive route for the production of butadiene as there are lower concentrations of undesirable compounds that need to be removed, such as isobutylene or isobutane. This reduces separation costs due to fewer similar boiling point compounds. The oxidative dehydrogenation process generates a crude butadiene stream having a high concentration of 1,3 butadiene, but also a high concentration of oxygenates relative to a typical steam cracker crude C4 stream.

The oxidative dehydrogenation process has some selectivity for the production of isobutylene. The isobutylene is removed to produce a purified butadiene process stream. Removal of the isobutylene is usually performed with an (methyl tertiary butyl ether) MTBE unit in a C4 complex. The removal of isobutylene can also be performed with an equivalent unit for the production of ETBE, or other similar processes. A major contaminant in the crude butadiene stream is furan, which can be as high as 2500 ppm by weight of the stream. The furan needs to be reduced to a safe level to protect downstream catalysts, including before the butadiene stream reaches the MTBE unit, but is not removed in a sodium bisulfate wash. The crude butadiene stream requires additional processing to remove heterocyclic oxygenate compounds generated by the oxidative dehydrogenation process. In particular, the heterocyclic oxygenate compounds includes furan, as the furan creates increased complexity and equipment and processing costs associated with the methanol recovery section in the MTBE unit. The furan also needs to be removed to prevent any furan contamination in the MTBE product stream. The furan contamination would render the MTBE product unstable which can result in peroxide formation and require the addition of inhibitors that are normally not required. Therefore, in order to meet MTBE product specifications, the furan needs to be reduced to a level that minimizes cost for the MTBE unit. Furan also can create problems with the butadiene extraction unit where furan can accumulate in the circulating solvent. Without removal of the furan, there is additional increased complexity and cost associated with the butadiene extraction unit, as the butadiene extraction solvent needs to maintain an operational level of purity. The buildup of furan in the solvent would require the treatment of the extraction solvent.

The present process provides a method of removal of the furan to very low levels to meet downstream process specifications. The process, as shown in the FIGURE, includes mixing a crude butadiene stream 6 with a solvent stream 4 to generate a first process stream 8. The first process stream 8 is passed to an extraction unit 10 to generate a second process stream 12 having a reduced oxygenate content, and a third process stream 14 having an increased, or high, oxygenate content. The second stream 12 is passed to a selective hydrogenation unit 40 to generate a fourth process stream 42 with a reduced acetylenes content. The fourth process stream 42 is passed to a 1,3-butadiene separation unit 50 to recover a purified 1,3-butadiene product stream 52. The separation unit 50 generates a raffinate stream 54, called Raffinate-1, containing the remaining C4s in the process stream 42.

The acetylenes include any acetylenes generated by the oxidative dehydrogenation process, and include acetylene, vinyl acetylene, methyl acetylene and ethyl acetylene. Other acetylenes that are generated would be expected to also be selectively hydrogenated in the selective hydrogenation unit, or have been seen to be in concentrations low enough to not be a concern for this process.

The second process stream 12 will contain some oxygenates, typically aldehydes and the like, that are not removed in the extraction unit 10. The second process stream 12 can be further passed through a wash unit 30, prior to passing the second process stream 12 to the selective hydrogenation unit 40. An oxygenate depleted stream 32 is generated and passed with a hydrogen stream 34 to the selective hydrogenation unit 40. The wash unit 30 comprises a sodium bisulfate wash unit to remove residual aldehydes and ketones not removed by the extraction unit 10.

The extraction unit 10 is preferably liquid-liquid extraction column, wherein the furan is soluble in the solvent. A solvent for use in the extraction distillation column 10 is an alcohol. A preferred alcohol is methanol. The extraction distillation includes passing a solvent stream 28 to the extraction unit 10. A water stream 24 is passed to the extraction unit 10. The water is a co-solvent to force splitting out of a separate aqueous phase and to avoid a high methanol content in the crude butadiene stream 12 leaving the extraction unit 10. The water and solvent contacting the crude butadiene stream removes the heterocyclic oxygenates, and in particular furan. Additional oxygenates are also removed. The third process stream 14 is withdrawn from the extraction unit 10 and passed to a solvent recovery unit 20.

The solvent recovery unit 20 generates a bottoms stream 24 comprising water. The water is recycled to the extraction unit for reuse. The solvent recovery unit 20 also generates an intermediate stream 22 comprising the solvent, which is split into two portions. A first portion is passed to be mixed with the crude butadiene stream, and a second portion is passed to the extraction unit 10. The solvent recovery unit 20 generates an overhead stream 26 comprising oxygenates, including furan, that have been removed from the crude butadiene stream. In the instant case, the solvent of choice is methanol. When another solvent is chosen, the solvent recovery unit can be configured to accommodate that solvent, including having the solvent passed out with the bottoms stream, and the bottoms stream passed to a separate distillation column for the separation of water and solvent.

The extraction unit 10 and the solvent recovery unit 20 provide for a closed loop system with respect to the water and methanol. The water and methanol cycle between the two units and under ideal operating conditions no new solvent needs to be added. Makeup solvent can be added due to normal losses during operation.

The process can further include passing the 1,3-butadiene stream with the oxygenates removed to an extractive distillation column to generate an overhead stream comprising the remaining C4s and a bottoms stream comprising 1,3-butadiene. Additional processing can separate out 1-butene from the remaining C4s, as 1-butene is a commercially valuable product. The bottoms stream includes an extractive solvent and the bottoms stream is passed to another distillation column for separating the 1,3-butadiene from the extractive solvent. The extractive solvent is in then recycled to the extractive distillation column for separating 1,3-butadiene from the mixture.

In general, the crude butadiene stream has a high butadiene content. The process includes mixing the crude butadiene stream with a solvent. The mixing can be done with a static mixer or other type of mixer to provide a well mixed mixture passed to a counter-current liquid-liquid extractor. More solvent and water are added to insure the absorption of the heterocyclic oxygenates, including furan, into the solvent mixture and to force phase separation of an aqueous phase, with the aqueous phase containing the oxygenates removed from the butadiene stream.

In one embodiment, if the extraction of the furan is sufficient from the mixing of the solvent with the butadiene stream, the a mixer-settler system can be used rather than the counter-current extractor. The mixed stream is passed to a mixing-settling unit, where additional solvent and water are passed to the mixing-settling unit. The water generates an aqueous phase comprising the furan, and a non-aqueous phase comprising C4 hydrocarbon compounds without furan is generated. The non-aqueous phase is passed to a wash unit using sodium bisulfate to remove aldehydes and ketones to generate an oxygenate free C4 stream. The oxygenate free C4 stream is then passed to a 1,3-butadiene purification unit to generate a purified 1,3-butadiene stream.

The aqueous phase is passed to a solvent recovery distillation column to recover the solvent and water for reuse. The water and solvent are in a closed loop with no need for continuous addition of solvent or water. The use of methanol as the solvent is preferred.

While the invention has been described with what are presently considered the preferred embodiments, it is to be understood that the invention is not limited to the disclosed embodiments, but it is intended to cover various modifications and equivalent arrangements included within the scope of the appended claims.

1. A process for the purification of a crude butadiene stream, comprising:
mixing the crude butadiene stream with a solvent to generate a first process stream;
passing the first process stream to an extraction unit to generate a second process stream with reduced oxygenate content and a third process stream with a high oxygenate content;
passing the second process stream to a selective hydrogenation unit to generate a fourth process stream with reduced acetylenes content; and
passing the fourth process stream to a 1,3-butadiene separation unit.

2. The process of claim 1 wherein the solvent is methanol.

3. The process of claim 1 further comprising:
adding more solvent to the extraction unit:
adding water to the extraction unit; and
withdrawing the third process stream.

4. The process of claim 3 further comprising:
passing the third process stream to a fractionation column to generate a bottoms stream comprising water, an intermediate stream comprising solvent, and an overhead stream comprising furan.

5. The process of claim 4 further comprising:
passing the bottoms stream to the extraction unit; and
passing the intermediate stream to the extraction unit.

6. The process of claims 1 further comprising:
passing the second process stream to a wash unit to remove residual oxygenates and generate an oxygenate depleted second process stream; and
passing the oxygenate depleted second process stream to the selective hydrogenation unit.

7. The process of claim 6 wherein the wash unit comprises a sodium bisulfate wash unit for removing residual aldehydes and ketones.

8. The process of claim 1 wherein the crude butadiene stream comprises heterocyclic oxygenate compounds.

9. The process of claim 8 wherein the heterocyclic oxygenate compounds include furan.

10. The process of claim 1 wherein the extraction unit is an extractive distillation column.

11. A process for the purification of 1,3-butadiene, comprising:
passing a crude butadiene stream, wherein the crude butadiene stream comprises heterocyclic oxygenates, to a mixer;
passing a solvent to the mixer to generate a first process stream comprising crude butadiene and solvent;
passing the first process stream to an extraction unit to generate a second process stream comprising 1,3-butadiene and having a reduced oxygenate content, and a third process stream comprising oxygenates;
passing the second process stream to a wash unit to generate an oxygenate depleted second process stream;
passing the oxygenate depleted second process stream to a selective hydrogenation unit to generate a fourth process stream;
passing the third process stream to a fractionation column to generate an overhead stream comprising heterocyclic oxygenate compounds, an intermediate stream comprising solvent, and a bottoms stream comprising water;
passing the bottoms stream to the extraction unit;
splitting the intermediate stream into a first portion and a second portion;
passing the first portion comprising solvent to the mixer;
passing the second portion comprising solvent to the extraction unit; and
passing the fourth process stream to a 1,3-butadiene separation unit to generate a purified 1,3-butadiene stream.

12. The process of claim 11 wherein the generation of the purified 1,3-butadiene stream comprises:
passing the fourth process stream to an extractive distillation column to generate an overhead stream comprising C4s, and a bottoms stream comprising 1,3-butadiene and an extractive solvent; and
passing the bottoms stream to a butadiene distillation column to generate an overhead stream comprising 1,3-butadiene and a bottoms stream comprising the extractive solvent.

13. The process of claim 11 wherein the heterocyclic oxygenate compound comprises furan.

14. The process of claim 11 wherein the wash unit comprises a sodium bisulfate wash unit.

15. The process of claim 11 wherein the solvent comprises an alcohol.

16. The process of claim 15 wherein the solvent comprises methanol.

17. A process for the removal of furan from a crude butadiene stream, comprising:
mixing the crude butadiene stream with a solvent in a mixer to generate a mixed stream;
passing the mixed stream to a liquid-liquid separation unit;
passing water to liquid-liquid separation unit, to generate a two phase system with an aqueous phase comprising water, solvent and furan, and a non-aqueous phase comprising C4 compounds, wherein the liquid-liquid separation unit is selected from the group consisting of a counter-current extraction column and a mixer-settler; and
drawing off the aqueous phase to recover the solvent and water.

18. The process of claim 17 wherein the solvent is methanol.

19. The process of claim 17 further comprising:
passing the aqueous phase to an distillation column to generate an overhead stream comprising furan, an intermediate stream comprising solvent, and a bottoms stream comprising water; and
passing the bottoms stream to the liquid-liquid separation unit; and
passing the intermediate stream to the mixer.

20. The process of claim 19 wherein the intermediate stream is split into a first portion and a second portion, and further comprising:
passing the first portion to the mixer; and
passing the second portion to the liquid-liquid separation unit.
